SEQUENCE LISTING (20 sequences)

SEQ ID NO: 1 - DNA, 20 bp, Artificial Sequence
  PlyV12 insert forward primer
ctgaacggtggttctacccc

SEQ ID NO: 2 - DNA, 21 bp, Artificial Sequence
  PlyV12 insert reverse primer
tttgaaggtaccccacgcttc

SEQ ID NO: 3 - DNA, 47 bp, Artificial Sequence
  PlyV12 P10N-V12C forward primer
cgtccgccgtacgaaaaagataccccgctgaacggtggttctacccc

SEQ ID NO: 4 - DNA, 51 bp, Artificial Sequence
  PlyV12 P10N-V12C reverse primer
ggatcctcaatggtggtgatgatggtgcgctttgaaggtaccccacgcttc

SEQ ID NO: 5 - DNA, 16 bp, Artificial Sequence
  LysEF-P10 insert forward primer
gcgccgaaaccgccgg

SEQ ID NO: 6 - DNA, 27 bp, Artificial Sequence
  LysEF-P10 insert reverse primer
cgcaactttgaactgcgggtgagacag

SEQ ID NO: 7 - DNA, 46 bp, Artificial Sequence
  LysEF-P10 V12N-P10C forward primer
gtatagcatgggttggtacgtttatcgtctggcgccgaaaccgccg

SEQ ID NO: 8 - DNA, 53 bp, Artificial Sequence
  LysEF-P10 V12N-P10C reverse primer
ggatcctcaatggtggtgatgatggtgcgcaactttgaactgcgggtgagaca

SEQ ID NO: 9 - protein, 338 aa, Artificial Sequence
  Lysin LKCHAPV12 amino acid sequence
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGSSGLNGGSTPPKPNTKKVKVLKHATNWSPSSKGAKMASFVKGGTFEVKQQRPISYSYSNQEYLIVNKGTVLGWVLSQDIEGGYGSDRVGGSKPKLPAGFTKEEATFINGNAPITTRKNKPSLSSQTATPLYPGQSVRYLGWKSAEGYIWIYATDGRYIPVRPVGKEAWGTFK

SEQ ID NO: 10 - DNA, 1014 bp, Artificial Sequence
  Lysin LKCHAPV12 nucleotide sequence
atggctaaaacccaggctgaaattaacaaacgtctggatgcgtacgcaaaaggtactgttgattccccgtatcgtgttaaaaaagcgaccagctacgatccgtccttcggtgttatggaagcgggtgcgatcgatgcagacggctactaccacgcacagtgtcaggatctgatcaccgattatgttctgtggctgaccgataacaaagttcgtacctggggtaacgcgaaagatcagatcaaacagtcttacggcaccggtttcaaaatccatgaaaacaaaccgagcaccgttccgaaaaaaggctggattgcggtttttacttctggctcttacgaacagtggggtcacatcggtatcgtttatgatggtggtaacaccagcaccttcaccattctggaacagaactggaacggttatgcgaacaaaaaaccgaccaaacgtgttgataactattatggtctgacccacttcattgaaatcccggttaaagcgggctcttctggtctgaacggtggtagcaccccgccgaaaccgaacaccaaaaaagttaaagttctgaaacatgcaaccaactggagcccgtctagcaaaggcgcgaaaatggcatccttcgttaaaggcggcacctttgaagttaaacagcagcgtccgatcagctactcttatagcaaccaggaatacctgatcgttaacaaaggtactgttctgggctgggttctgagccaggatattgaaggtggttacggttccgatcgtgttggtggttctaaaccgaaactgccggcgggtttcaccaaagaagaagcgaccttcattaacggtaacgcgccgatcactacccgtaaaaataaaccgagcctgagttctcagaccgcgaccccgctgtatccgggtcagtctgttcgttatctgggctggaaatctgcggaaggttacatctggatctacgcgaccgatggtcgttatatcccggttcgtccggttggtaaagaagcgtggggcaccttcaaa

SEQ ID NO: 11 - protein, 321 aa, Artificial Sequence
  Lysin P10N-V12C amino acid sequence
MVKVNDVVSYLNSRVGTGIDMDGAYGFQCADLAQAITYNFFGWWFYGNAIALASQPIPNGFERIRVTDATQIKAGDIVVWSEHEYAQYGHVAIAAKDGYSDQTFENYAQNWLNASLTVGSPIALVRTNMYGVGYVIRPPYEKDTPLNGGSTPPKPNTKKVKVLKHATNWSPSSKGAKMASFVKGGTFEVKQQRPISYSYSNQEYLIVNKGTVLGWVLSQDIEGGYGSDRVGGSKPKLPAGFTKEEATFINGNAPITTRKNKPSLSSQTAIPLYPGQSVRYLGWKSAEGYIWIYATDGRYIPVRPVGKEAWGTFKAHHHHHH

SEQ ID NO: 12 - DNA, 963 bp, Artificial Sequence
  Lysin P10N-V12C nucleotide sequence
atggttaaagttaacgatgttgttagctacctgaactctcgtgttggtaccggtatcgatatggatggtgcgtacggtttccagtgcgcggatctggcgcaggcgatcacctacaacttcttcggttggtggttctacggcaacgcgatcgcactggcgtctcagccgatcccgaacggcttcgaacgtatccgtgttaccgatgcgacccagatcaaagcgggcgatatcgttgtttggagcgaacacgaatacgcgcagtacggtcacgttgcgatcgcggcgaaagatggctacagcgatcagaccttcgaaaactacgcgcagaactggctgaacgcgagcctgaccgttggtagcccgatcgcgctggttcgtaccaacatgtacggcgttggttacgttatccgtccgccgtacgaaaaagataccccgctgaacggtggttctaccccgcctaaaccgaacactaaaaaagttaaagttctgaaacatgcaaccaactggtctccgagctctaaaggtgcgaaaatggctagcttcgttaaaggcggtactttcgaagttaaacagcagcgtccgattagctactcttactctaaccaggaatacctgattgttaacaaaggtaccgttctgggttgggttctgagccaggatattgaaggtggttacggctctgatcgtgttggcggtagcaaaccgaaactgccggctggtttcaccaaagaagaagcgaccttcatcaacggtaacgctccgatcaccacccgtaaaaacaaaccgtctctgtctagccagaccgcgatcccgctgtatccgggtcagagcgttcgttacctgggctggaaatctgctgaaggttacatctggatctacgcgaccgatggtcgttacatcccggttcgtccggttggtaaagaagcgtggggtaccttcaaagcgcaccatcatcaccaccat

SEQ ID NO: 13 - protein, 242 aa, Artificial Sequence
  Lysin V12NM3 amino acid sequence
MSNINMETAIANMYALKARGITYSMNYSRTGADGTGDCSGTVYDSLRKAGASDAGWVLNTDSMHSWLEKNGFKLIAQNKEWSAKRGDVVIFGKKGASGGSAGHVVIFISSTQIIHCTWKSATANGVYVDNEATTCPYSMGWYVYRGSSGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 14 - DNA, 726 bp, Artificial Sequence
  Lysin V12NM3 nucleotide sequence
atgtctaacattaacatggaaaccgcgatcgctaacatgtatgctctgaaagcgcgtggtatcacctactctatgaactacagccgtaccggtgctgatggcaccggtgattgttctggcaccgtttacgattctctgcgtaaagcaggtgcttctgatgcgggttgggttctgaacaccgatagcatgcacagctggctggagaaaaacggcttcaaactgattgctcagaacaaagaatggtctgcaaaacgtggtgatgttgttatctttggtaaaaaaggtgcatctggtggcagcgcgggtcacgttgttatcttcatcagctccacccagatcattcattgcacctggaaaagcgctaccgcgaacggtgtttacgttgataacgaagctaccacctgtccgtactctatgggttggtacgtttaccgtggctcttctggtaaatctgcgagcaaaatcaccgttggttctaaagcgccgtacaacctgaaatggtctaaaggtgcgtacttcaacgcaaaaatcgatggtctgggtgcgacctctgcgacccgttacggtgataaccgtaccaactaccgtttcgatgttggtcaggcggtttacgcgccgggcaccctgatctacgttttcgaaatcatcgatggttggtgccgtatctattggaacaaccacaacgaatggatctggcacgaacgtctgatcgttaaagaagttttc

SEQ ID NO: 15 - protein, 262 aa, Artificial Sequence
  Lysin LKNM3 amino acid sequence
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGSSGKSASKITVGSKAPYNLKWSKGAYFNAKIDGLGATSATRYGDNRTNYRFDVGQAVYAPGTLIYVFEIIDGWCRIYWNNHNEWIWHERLIVKEVF

SEQ ID NO: 16 - DNA, 786 bp, Artificial Sequence
  Lysin LKNM3 nucleotide sequence
atggctaaaacccaggctgaaattaacaaacgtctggatgcgtacgcaaaaggtactgttgattccccgtatcgtgttaaaaaagcgaccagctacgatccgtccttcggtgttatggaagcgggtgcgatcgatgcagacggctactaccacgcacagtgtcaggatctgatcaccgattatgttctgtggctgaccgataacaaagttcgtacctggggtaacgcgaaagatcagatcaaacagtcttacggcaccggtttcaaaatccatgaaaacaaaccgagcaccgttccgaaaaaaggctggattgcggtttttacttctggctcttacgaacagtggggtcacatcggtatcgtttatgatggtggtaacaccagcaccttcaccattctggaacagaactggaacggttatgcgaacaaaaaaccgaccaaacgtgttgataactattatggtctgacccacttcattgaaatcccggttaaagcgggttcttctggtaaatctgcgagcaaaatcaccgttggttctaaagcgccgtacaacctgaaatggtctaaaggtgcgtacttcaacgcaaaaatcgatggtctgggtgcgacctctgcgacccgttacggtgataaccgtaccaactaccgtttcgatgttggtcaggcggtttacgcgccgggcaccctgatctacgttttcgaaatcatcgatggttggtgccgtatctattggaacaaccacaacgaatggatctggcacgaacgtctgatcgttaaagaagttttc

SEQ ID NO: 17 - protein, 321 aa, Artificial Sequence
  Lysin PlyV12 amino acid sequence
MSNINMETAIANMYALKARGITYSMNYSRTGADGTGDCSGTVYDSLRKAGASDAGWVLNTDSMHSWLEKNGFKLIAQNKEWSAKRGDVVIFGKKGASGGSAGHVVIFISSTQIIHCTWKSATANGVYVDNEATTCPYSMGWYVYRLNGGSTPPKPNTKKVKVLKHATNWSPSSKGAKMASFVKGGTFEVKQQRPISYSYSNQEYLIVNKGTVLGWVLSQDIEGGYGSDRVGGSKPKLPAGFTKEEATFINGNAPITTRKNKPSLSSQTATPLYPGQSVRYLGWKSAEGYIWIYATDGRYIPVRPVGKEAWGTFKAHHHHHH

SEQ ID NO: 18 - DNA, 963 bp, Artificial Sequence
  Lysin PlyV12 nucleotide sequence
atgtctaacatcaacatggaaaccgcaatcgctaacatgtacgcgctgaaagctcgtggtattacctatagcatgaactatagccgtaccggtgctgatggtaccggtgattgctctggcaccgtgtacgatagcctgcgtaaagcgggcgctagcgatgctggctgggttctgaacaccgattctatgcattcctggctggaaaagaacggctttaaactgatcgcacagaacaaagaatggtctgctaaacgtggtgacgttgttattttcggtaaaaaaggtgctagcggcggtagcgctggccacgttgttatcttcatcagcagcacccagattatccactgtacctggaaatctgcgaccgcgaacggtgtttacgttgataacgaagctaccacctgcccgtatagcatgggttggtacgtttatcgtctgaacggtggttctaccccgcctaaaccgaacactaaaaaagttaaagttctgaaacatgcaaccaactggtctccgagctctaaaggtgcgaaaatggctagcttcgttaaaggcggtactttcgaagttaaacagcagcgtccgattagctactcttactctaaccaggaatacctgattgttaacaaaggtaccgttctgggttgggttctgagccaggatattgaaggtggttacggctctgatcgtgttggcggtagcaaaccgaaactgccggctggtttcaccaaagaagaagcgaccttcatcaacggtaacgctccgatcaccacccgtaaaaacaaaccgtctctgtctagccagaccgcgaccccgctgtatccgggtcagagcgttcgttacctgggctggaaatctgctgaaggttacatctggatctacgcgaccgatggtcgttacatcccggttcgtccggttggtaaagaagcgtggggtaccttcaaagcgcaccatcatcaccaccat

SEQ ID NO: 19 - protein, 260 aa, Artificial Sequence
  Lysin LysEF-P10 amino acid sequence
MHHHHHHSSGVDLGTENLYFQSMVKVNDVVSYLNSRVGTGIDMDGAYGFQCADLAQAITYNFFGWWFYGNAIALASQPIPNGFERIRVTDATQIKAGDIVVWSEHEYAQYGHVAIAAKDGYSDQTFENYAQNWLNASLTVGSPIALVRTNMYGVGYVIRPPYEKDTPAPKPPAPKPAKPNLPTPKGDDTMLAIYYKHLKNGNVEQWLLINGKRVYLPTQTWVNEANALIKAYGGTKEVVQYNHDNFGLKLLELSHPQFKV

SEQ ID NO: 20 - DNA, 780 bp, Artificial Sequence
  Lysin LysEF-P10 nucleotide sequence
atgcaccatcatcatcatcattcttctggtgtagatctgggtaccgagaacctgtacttccaatccatggttaaagttaacgatgttgttagctacctgaactctcgtgttggtaccggtatcgatatggatggtgcgtacggtttccagtgcgcggatctggcgcaggcgatcacctacaacttcttcggttggtggttctacggcaacgcgatcgcactggcgtctcagccgatcccgaacggcttcgaacgtatccgtgttaccgatgcgacccagatcaaagcgggcgatatcgttgtttggagcgaacacgaatacgcgcagtacggtcacgttgcgatcgcggcgaaagatggctacagcgatcagaccttcgaaaactacgcgcagaactggctgaacgcgagcctgaccgttggtagcccgatcgcgctggttcgtaccaacatgtacggcgttggttacgttatccgtccgccgtacgaaaaagataccccggcgccgaaaccgccggctccgaaaccggctaaaccgaacctgccgaccccgaaaggtgatgataccatgctggctatctattacaaacatctgaaaaacggtaacgttgaacagtggctgctgattaacggtaaacgcgtttacctgccgacccagacctgggttaacgaagcgaacgctctgattaaagcgtatggtggcaccaaagaagttgttcagtataaccatgataacttcggcctgaaactgctggaactgtctcacccgcagttcaaagtt

(19) United States
(12) Patent Application Publication
(10) Pub. No.: US 2023/0416719 A1
(43) Pub. Date: Dec. 28, 2023

(54) ENGINEERED CHIMERIC ANTIMICROBIAL AGENTS AGAINST GRAM-POSITIVE BACTERIA

(71) Applicant: Massachusetts Institute of Technology, Cambridge, MA (US)

(72) Inventor: Boon Chong GOH, Singapore (SG)

(21) Appl. No.: 18/252,865

(22) PCT Filed: Nov. 14, 2021
(86) PCT No.: PCT/US2021/059290

§ 371 Date: May 12, 2023

(30) Foreign Application Priority Data
Nov. 13, 2020 (SG) 1020201132IV

(51) Int. Cl. C12N 9/80; C07K 14/005; A01N 63/50; A61P 31/04; A01P 1/00

(52) U.S. Cl. CPC C12N 9/80; C07K 14/005; A01N 63/50; A61P 31/04; A01P 1/00; C12N 2795/10022; C12N 2795/10031; C12N 2795/10033; C12N 2795/10122; C12N 2795/10131; C12N 2795/10133; C07K 2319/33

(57) ABSTRACT

The present invention relates to chimeric bacteriophage lysins which can be used to sanitize surfaces, for the treatment of bacterial infections and/or for the selective treatment of body odor-causing bacteria. The invention also includes synergistic combinations, methods of treatment and isolated polynucleotides which encode the chimeric lysins.

### CROSS-REFERENCE TO RELATED APPLICATIONS

The present application is a filing under 35 U.S.C. 371 as the National Stage of International Application No. PCT/US2021/059290, filed Nov. 14, 2021, entitled “ENGINEERED CHIMERIC ANTIMICROBIAL AGENTS AGAINST GRAM-POSITIVE BACTERIA,” which claims priority to Singapore Application No. SG 10202011321V filed with the Intellectual Property Office of Singapore on Nov. 13, 2020, both of which are incorporated herein by reference in their entirety for all purposes.

### INCORPORATION BY REFERENCE OF MATERIAL IN ASCII TEXT FILE

This application incorporates by reference the Sequence Listing contained in the following ASCII text file being submitted concurrently herewith:

File name: 4373-18800 SP103343USZBD Sequence Listing ST25; created on May 11, 2023; and having a file size of 25 KB.

The information in the Sequence Listing is incorporated herein in its entirety for all purposes.

### FIELD OF THE INVENTION

The present invention relates to chimeric bacteriophage lysins which can be used to sanitize surfaces, for the treatment of bacterial infections and/or for the selective treatment of body odor-causing bacteria. The invention also includes synergistic combinations, isolated polynucleotides which encode the chimeric lysins.

### BACKGROUND OF THE INVENTION

Antimicrobial resistance (AMR) is on the rise worldwide, which poses a public health crisis given the slow pace of antibiotic development. To treat infections caused by drug-resistant bacteria, health care providers are resorting to mothballed antibiotics that come with serious side effects [Velez, R. and Sloand, E., J Clin Nurs 25: 1886-1889 (2016); Ventola, C. L. P T 40: 277-83 (2015)]. Therefore, there is a dire need to develop novel antibacterial agents. Lysins derived from bacteriophage present a promising alternative to antibiotics as they are less prone to resistance and can kill bacteria selectively and rapidly [Fischetti, V. A., Curr Opin Microbiol 11: 393-400 (2008); Fischetti, V. A., Viruses 10 (2018); Schmelcher, M. et al., Future Microbiol 7: 1147-1171 (2012); Zou, G. et al., J Biol Chem 286: 14362-14372 (2011); Maciejewska, B. et al., Appl Microbiol Biotechnol 102: 2563-2581 (2018); Oliveira, H. et al., Viruses 10 (2018)].

Lysins are phage-encoded peptidoglycan hydrolases that cleave the bonds in peptidoglycan chains of the bacteria outer cell wall. When applied exogenously to bacteria as purified recombinant proteins, lysins rapidly hydrolyze the peptidoglycan layer, resulting in cell lysis and bacterial death. Hence, lysins are particularly effective against Gram-positive bacteria because of the thick peptidoglycan layer on the bacterial surface [Fischetti, V. A., Curr Opin Microbiol 11: 393-400 (2008); Hermoso, J. A. et al., Curr Opin Microbiol 101-472 (2007); Fischetti, V. A. Int J Med Microbiol 300: 357-362 (2010)]. Lysins targeting Gram-positive bacteria contains two functional, modular domains, namely catalytic domain (CD) and cell wall binding domain (CBD). CD cleaves the specific bonds of the bacterial peptidoglycan, and CBD recognizes the target bacteria and anchors the lysin by binding to specific carbohydrate ligands on the bacterial cell wall, contributing to the selective nature of lysine [Loessner, M. J. et al., Mol Microbiol 44: 335-349 (2002); Lopez, R. et al., Microb Drug Resist 3: 199-211 (1997)].

The lytic spectrum of a lysin ranges from species-specific to targeting multiple genera. There are lysins that specifically target a single bacterial species (e.g., LysEF-P10 for E. faecalis [Cheng, M. et al., Sci Rep 7: 1-15 (2017)], and PlyPSA for Listeria monocytogenes [Schmelcher, M. et al., Microb Biotechnol 4: 651-662 (2011); Zimmer, M. et al., Mol Microbiol 50: 303-317 (2003)]) while some target various bacterial species of the same genus (e.g. LysK [O'Flaherty, S. et al., J Bacteriol 187: 7161-7164 (2005)]). Interestingly, several lysins have been identified to target multiple genera [Deutsch, S.-M. et al., Appl Environ Microbiol 70: 96-103 (2004); Yoong, P. et al., J Bacteriol 186: 4808-4812 (2004); Gilmer, D. B. et al., Antimicrob Agents Chemother 57: 2743-2759 (2013)]. For example, PlyV12 targets enterococci, staphylococci, and streptococci [Yoong, P. et al., J Bacteriol 186: 4808-4812 (2004)], and PlySs2 lyses staphylococci, streptococci and listeria [Gilmer, D. B. et al., Antimicrob Agets Chemother 57: 2743-2759 (2013)].

The modular architecture of Gram-positive lysin enables the construction of chimeric lysins with enhanced lytic spectrum and lytic activity [Loessner, M. J. et al., Mol Microbiol 44: 335-349 (2002); Schmelcher, M. et al., Microb Biotechnol 4: 651-662 (2011); Yang, H. et al., Front Microbiol 5: 1-(2014); Dong, Q. et al., Microb Biotechnol 8: 210-220 (2015); Broendum, S. S. et al., Mol Microbiol 110: 879-896 (2018)]. For example, a chimeric lysin Ply187N-V12C exhibited a broader lytic spectrum from only targeting staphylococci to also kill enterococci and streptococci after adding the CBD of PlyV12 [Dong, Q. et al., Microb Biotechnol 8: 210-220 (2015)]. While the CBD largely determines the lytic spectrum by recognizing specific cell wall components on the bacteria, the CD dictates the type of peptidoglycan bond to be cleaved [Broendum, S. S. et al., Mol Microbiol 110: 879-896 (2018); Oliveira, H. et al., J Virol 87: 4558-4570 (2013); Nelson, D. C. et al., Advances in Virus Research (2012); Diez-Martinez, R. et al., Antimicrob Agents Chemother 57: 5355-5365 (2013)]. The dependence of CBD for target recognition can be circumvented by tweaking the net charge of a lysin. A positively-charged CD-only lysin was shown to maintain its lytic activity while possessing a broader lytic spectrum [Low, L. Y. et al., J Biol Chem 286: 34391-34403 (2011)]. Bactericidal activity can be improved by fusing the CBD of a streptococcal lysin with the CD of a different lysin, or by modifying the linker between CD and CBD [Yang, H. et al., Antimicrob Agents Chemother 63 (2019); Yang, H. et al., Antimicrob Agents Chemother 64 (2020)].

While there are many reports that successfully extended the lytic spectrum of a lysin [Schmelcher, M. et al., Microb Biotechnol 4: 651-662 (2011); Dong, Q. et al., Microb Biotechnol 8: 210-220 (2015); Broendum, S. S. et al., Mol Microbiol 110: 896 (2018); Diez-Martinez, R. et al., Antimicrob Agents Chemother 57: 5355-5365 (2013)], few have managed to introduce species specificity in a chimeric lysin.

A promising application of lysins is on the human skin, particularly to eradicate the problem of body odor (BO). The formation of body odors (BO) is caused by factors such as diet, gender, health, and medication, but the major contribution comes from bacterial activity on skin gland secretions. Staphylococcus hominis, Staphylococcus haemolyticus, Staphylococcus epidermidis, and Corynebacterium jeikeium are often associated with producing BO in human skin, particularly in the armpits. BO gases are produced when these BO-causing bacteria degrade the amino acids and fatty acids found in sweat.

Current anti-BO solutions in the market can be categorized into two types: antiperspirant, which reduces sweat production, and deodorant, which masks odor. None of the existing solutions target the underlying cause of BO, i.e., bacteria.

There is a need for alternative solutions that target problematic bacteria.

### SUMMARY OF THE INVENTION

Chimeric lysins were created by interchanging the CBDs of parent lysins. The lytic spectra of these chimeric lysins were characterized by subjecting them to a panel of enterococcal and staphylococcal strains under various conditions. Certain chimeric lysins are useful for various infections, whereas particular chimeric lysins have been developed for a new treatment to selectively eliminate BO-causing bacteria. The key advantages of lysins include their good safety profile, as lysins are not systemically absorbed, and are biodegradable.

In a first aspect the invention provides a chimeric bacteriophage lysin comprising a catalytic domain of a first phage lysin and a binding domain of a second phage lysin, wherein the chimeric lysin has killing activity against a plurality of Staphylococcus species, wherein the lysin comprises an amino acid sequence set forth in:
a) SEQ ID NO: 11, or a variant thereof wherein said variant is capable of killing staphylococci;
b) amino acids 1-165 and 170-338 set forth in SEQ ID NO: 9, or a variant thereof wherein said variant is capable of killing staphylococci;
c) amino acids 1-145 and 150-242 set forth in SEQ ID NO: 13, or a variant thereof wherein said variant is capable of killing staphylococci;
d) amino acids 1-165 and 170-262 set forth in SEQ ID NO: 15, or a variant thereof wherein said variant is capable of killing staphylococci;

In some embodiments the chimeric bacteriophage lysin variant comprises a sequence having at least 85%, at least 90% or at least 95% sequence identity to SEQ ID NO: 11, SEQ ID NO: 9, SEQ ID NO: 13, or SEQ ID NO: 15.

In some embodiments, the said first and second binding domains of the chimeric lysin of 1 b), c) or d) are fused by a linker peptide.

In some embodiments, the linker peptide has the amino acid sequence set forth in amino acids 166-169 of SEQ ID NO: 9.

In some embodiments of:
a) the chimeric lysin has the amino acid sequence set forth in SEQ ID NO: 11;
b) the chimeric lysin has the amino acid sequence set forth in SEQ ID NO: 9;
c) the chimeric lysin has the amino acid sequence set forth in SEQ ID NO: 13;
d) the chimeric lysin has the amino acid sequence set forth in SEQ ID NO: 15.

In some embodiments:
i) the chimeric lysin set forth in a) also has killing activity against E. faecalis and/or S. hominis and S. epidermidis in human sweat;
ii) the chimeric lysin set forth in b) has killing activity against S. hominis and S. epidermidis in human sweat;
iii) the chimeric lysin set forth in d) has killing activity against S. epidermidis in human sweat.

In some embodiments, the chimeric lysin in a) has killing activity against methicillin-resistant and methicillin-sensitive strains of S. aureus.

In some embodiments, the chimeric lysin in a) has killing activity against Rifampicin resistant strains of E. faecalis and Vancomycin resistant strains of E. faecalis.

In some embodiments, the chimeric lysin in a) has killing activity over a pH range of about 4-10 and/or over a salt concentration of about 0-500 mM NaCl.

In a second aspect, the invention provides an isolated polynucleotide molecule encoding a chimeric lysin defined in the first aspect.

In some embodiments of the isolated polynucleotide molecule:
i) the nucleic acid sequence in a) has at least 85% sequence identity to SEQ ID NO: 12;
ii) the nucleic acid sequence in b) has at least 85% sequence identity to SEQ ID NO: 10;
iii) the nucleic acid sequence in c) has at least 85% sequence identity to SEQ ID NO: 14;
iv) the nucleic acid sequence in d) has at least 85% sequence identity to SEQ ID NO: 16; due to the degeneracy of the genetic code.

In a third aspect, the invention provides a composition comprising one or more chimeric lysins of the first aspect. Preliminary studies have shown potential synergistic effect by using two different lysins. It has also been found that a combination of a chimeric lysin such as LKCHAPV12 (having the amino acid sequence set forth in SEQ ID NO: 9) and native lysin such as PlyV12 (having the amino acid sequence set forth in SEQ ID NO: 17, and the nucleic acid sequence set forth in SEQ ID NO: 18) can act synergistically in killing Staphylococci (Table 4). LysEF-P10 (having the amino acid sequence set forth in SEQ ID NO: 19, and the nucleic acid sequence set forth in SEQ ID NO: 20) is a native lysin that may be combined with a chimeric lysin for synergistic effect.

In some embodiments, the composition is a synergistic composition comprising one or more chimeric lysins and one or more native lysins.

In some embodiments, the composition is for sanitizing or decontaminating porous or non-porous surfaces comprising at least one chimeric bacteriophage lysin of any aspect of the invention or the composition of the third aspect.

In some embodiments, the composition is for medical or cosmetic use.

In some embodiments the composition further comprises an antibiotic.

In some embodiments, the composition is formulated for administration to the skin.

Suitable formulations would be well-known to those skilled in the art. Formulations may include chimeric bacteriophage lysins of any aspect of the invention encapsulated within lipid/polymer nano or microparticles.

In a fourth aspect, the invention provides use of a composition of the third aspect for the manufacture of a medicament for the prophylaxis or treatment of a staphylococcal infection.

In some embodiments, the medicament is for the prophylaxis or treatment of body odor.

In some embodiments, the medicament comprises at least one chimeric lysin selected from the group comprising the chimeric lysin defined in a) and the chimeric lysin defined in b) of the first aspect.

In some embodiments, the medicament is for the prophylaxis or treatment of an E. faecalis infection.

In a fifth aspect, the invention provides a method of prophylaxis or treatment of a staphylococcal infection in a mammal, comprising administering to said mammal one or more chimeric lysins of the first aspect or a composition of the third aspect.

In some embodiments, the staphylococcal infection causes body odor in a human and the one or more chimeric lysins are selected from any one of a), b), or d) of the first aspect.

In a sixth aspect, the invention provides a method for decontaminating inanimate surfaces suspected of containing infectious bacteria, comprising treatment of said surfaces with a bactericidal or bacteriostatic effective amount of at least one chimeric bacteriophage lysin of any aspect of the invention or the composition of the third aspect.

### BRIEF DESCRIPTION OF THE FIGURES

FIG. 1A and FIG. 1B show a description of the parent and chimeric recombinant lysins. (A) Schematic diagram illustrating the domain swapping between the parent lysins to create the two chimeras. The N-terminal domain is the catalytic domain whereas the C-terminal domain is the cell-wall binding domain. (B) The SDS-PAGE analysis showed the high purity of the produced lysins. PlyV12 (35.0 kDa), P10N-V12C (35.4 kDa), V12N-P10C (27.2 kDa) contain a C-terminal 6×His tag, while LysEF-P10 (29.1 kDa) contains a N-terminal 6×His-TEV for affinity chromatography purification.

FIG. 2A and FIG. 2B show lytic activity characterization. (A) Killing kinetics of PlyV12 against E. faecalis OR1GF strain at various doses as measured by the reduction in OD₆₀₀. (B) Lytic activity at various doses as measured by the rate of reduction in OD₆₀₀ in the first 3 minutes. These experiments were performed in biological triplicates.

FIG. 3 shows the dose response for other 3 lysins. Lysin concentrations that achieved >90% OD₆₀₀ reduction were used in this study.

FIG. 4A and FIG. 4B show characterization of lysins. The lytic activities of the four lysins at various pH buffers (A) under different salt concentrations (B). These experiments were done in biological triplicates.

FIG. 5A, FIG. 5B, FIG. 5C, and FIG. 5D show lytic spectra of the parent and chimeric lysins. The activities of these four lysins against 12 enterococcal and 10 staphylococcal strains were measured as the reduction in OD₆₀₀ per minute in the first three minutes. These experiments were done in biological triplicates.

FIG. 6A, FIG. 6B, FIG. 6C, FIG. 6D, FIG. 6E, FIG. 6F, FIG. 6G, FIG. 6H, FIG. 6I, and FIG. 6J show the effect of lysin treatment on E. faecium. FSC-SSC dot plots of E. faecium cells upon treated with PlyV12 (A), P10N-V12C (B), and without lysin treatment (C). (D-F) The corresponding histogram displaying percentage of gated cells with PI uptake. PI-negative and PI-positive cells are labeled with R1 and R2 respectively. (G) The bactericidal activity of the two lysins on E. faecium. Confocal microscopy images of the E. faecium cells with treatment of PlyV12 (H), P10N-V12C (I) and without lysin treatment (J). Cells with PI uptake were detected as red fluorescence signals. White bars indicate 10 μm.

FIG. 7 shows controls for the flow cytometry experiments used to establish gating parameters. The FSC-SSC plots are at the top row, whereas the bottom row shows the percentage of gated cells with PI uptake. PI-negative and PI-positive cells are labeled with R1 and R2 respectively.

FIG. 8 shows SSC-FSC plots of the 4 replicates that were performed. PlyV12 and P10N-V12C lysin treated E faecium cells are shown in the top and middle rows, respectively. The untreated control runs are shown in the bottom row. BR1(1) refers to biological replicate 1, technical duplicate 1.

### DETAILED DESCRIPTION OF THE INVENTION

### Definitions

Certain terms employed in the specification, examples and appended claims are collected here for convenience.

As used in the specification and the appended claims, the singular forms “a,” “an” and “the” include plural referents unless the context clearly dictates otherwise.

The terms “amino acid” or “amino acid sequence,” as used herein, refer to an oligopeptide, peptide, polypeptide, or protein sequence, or a fragment of any of these, and to naturally occurring or synthetic molecules. Where “amino acid sequence” is recited herein to refer to an amino acid sequence of a naturally occurring protein molecule, “amino acid sequence” and like terms are not meant to limit the amino acid sequence to the complete native amino acid sequence associated with the recited protein molecule.

The term “variant” as used herein, refers to an amino acid sequence that is altered by one or more amino acids, but retains the ability of the non-variant reference sequence to bind to and kill staphylococci. The variant may have “conservative” changes, wherein a substituted amino acid has similar structural or chemical properties (e.g., replacement of leucine with isoleucine). More rarely, a variant may have “non-conservative” changes (e.g., replacement of glycine with tryptophan). Analogous minor variations may also include amino acid deletions or insertions, or both. Moreover, guidance in determining which amino acid residues may be substituted, inserted, or deleted without abolishing biological activity may be found using computer programs well known in the art, for example, DNASTAR® software (DNASTAR, Inc. Madison, Wisconsin, USA). A non-limiting example in the context of chimeric lysins of the invention is the presence of a peptide linker between the catalytic domain and the binding domain. In some embodiments the chimeric bacteriophage lysin variant comprises a sequence having at least 85%, at least 90% or at least 95% sequence identity to SEQ ID NO: 11, SEQ ID NO: 9, SEQ ID NO: 13, or SEQ ID NO: 15.

The phrases “nucleic acid” or “nucleic acid sequence,” as used herein, refer to an oligonucleotide, nucleotide, polynucleotide, or any fragment thereof, to DNA or RNA of genomic or synthetic origin which may be single-stranded or double-stranded and may represent the sense or the antisense strand, to peptide nucleic acid (PNA), or to any DNA-like or RNA-like material.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

Bibliographic references mentioned in the present specification are for convenience listed in the form of a list of references and added at the end of the examples. The whole content of such bibliographic references is herein incorporated by reference but their mention in the specification does not imply that they form part of the common general knowledge.

Having now generally described the invention, the same will be more readily understood through reference to the following examples which are provided by way of illustration, and are not intended to be limiting of the present invention.

### EXAMPLES

Standard molecular biology techniques known in the art and not specifically described were generally followed as described in Green and Sambrook, Molecular Cloning: A Laboratory Manual, Cold Springs Harbor Laboratory, New York (2012).

### Example 1

### Materials and Methods

### Cloning, Expression and Purification of Phage Lysins

Lysin genes PlyV12 (Genbank accession No. AAT01859.1) and LysEF-P10 (GenBank accession No. AQT27695.1) were synthesized and cloned into pNIC-CH and pNIC28-Bsa4 vectors by Bio Basic Inc. The nucleotide sequences were codon-optimized to improve the efficiency of soluble expression in E. coli. Chimeric lysin genes of P10N-V12C (SEQ ID NOs: 11 and 12) and V12N-P10C were produced using overlap extension PCR using the primers listed in Table 1.

TABLE 1
Primer designs to create chimeric lysins P10N-V12C and V12N-P10C

| Target gene | Primer | Sequence (5′ to 3′) | SEQ ID NO: |
|---|---|---|---|
| PlyV12 insert | plyV12 | CTGAACGGTGGTTCTACCCC | 1 |
| | fwd | | |
| | plyV12 rev | TTTGAAGGTACCCCACGCTTC | 2 |
| PlyV12 chimeric | P10N- | CGTCCGCCGTACGAAAAAGATACCCCGCTGA | 3 |
| insert | V12C fwd | ACGGTGGTTCTACCCC | |
| | P10N- | GGATCCTCAATGGTGGTGATGATGGTGCGCT | 4 |
| | V12C rev | TTGAAGGTACCCCACGCTTC | |
| LysEF-P10 | LysEF- | GCGCCGAAACCGCCGG | 5 |
| insert | P10 fwd | | |
| | LysEF- | CGCAACTTTGAACTGCGGGTGAGACAG | 6 |
| | P10 rev | | |
| LysEF-P10 | V12N- | GTATAGCATGGGTTGGTACGTTTATCGTCTG | 7 |
| chimeric insert | P10C fwd | GCGCCGAAACCGCCG | |
| | V12N- | GGATCCTCAATGGTGGTGATGATGGTGCGCA | 8 |
| | P10C rev | ACTTTGAACTGCGGGTGAGACA | |

To produce the 2 chimeric lysins, the C-terminal cell wall binding domain (CBD) of PlyV12 and LysEF-P10 was amplified as inserts for P10N-V12C and V12N-P10C respectively. The inserts were purified and extended using primers containing the sequences of the N-terminal CD and the C-terminal histag in the vector pNIC-CH. The extended PCR products will be used as megaprimers to swap the CBD of PlyV12 and LysEF-P10, creating the two chimeric lysins. After confirmed by sequencing, the plasmids were transformed into competent cells of BL21(DE3)-T1R E. coli Rosetta strain for efficient protein expression.

To produce the recombinant lysins, the transformed E. coli was incubated at 37° C. in LB broth containing 34 μg/ml chloramphenicol and 50 μg/ml kanamycin until the culture reaches mid-log phase (OD₆₀₀ 0.5-0.6). After cooling the culture down, protein overexpression was induced by adding 0.2 mM isopropyl β-D-thiogalactoside and incubated at 16° C. for 16-18 hours. Cells were harvested by centrifugation at 4,000 g for 25 min at 4° C. and the cell pellet was stored at −80° C. until ready to purify.

To purify the proteins, the cell pellets were resuspended with lysis buffer before being disrupted by sonication on ice. PlyV12, chimeric lysins P10N-V12C and V12N-P10C used lysis buffer at pH 7.5 (50 mM HEPES pH 7.5, 500 mM NaCl, 5% glycerol, 0.5 mM DTT), whereas LysEF-P10 used lysis buffer at pH 8.5 (50 mM Tris pH 8.5, 500 mM NaCl, 5% glycerol, 0.5 mM DTT). Cell debris was removed by centrifugation for 1 hour at 40,000×g at 4° C. to obtain the soluble protein. The supernatant was sterile filtered and mixed with 2 mL of nickel-nitrilotriacetic acid (Ni-NTA) resin and was incubated for 2 hours at 4° C. in a tube rotator. The desired proteins were collected by washing and eluting with lysis buffers containing 0, 10, 20, 250, or 500, mM imidazole. Finally, the collected fractions were subjected to buffer exchange to reduce the imidazole concentration to <10 mM and concentrated to >5 mg/ml before storing at −80° C.

### Characterization of Lytic Activities and Spectra of Recombinant Lysins.

Turbidity reduction assay was performed in 96-well microtiter plate to measure the lytic activity of the lysins produced [Nelson, D. et al., Proc Natl Acad Sci 98: 4107-4112 (2001); Yang, H. et al., Antimicrob Agents Chemother 58: 536-542 (2014)]. Enterococcal and staphylococcal strains were grown to mid-logarithmic phase in brain heart infusion (BHI) and Mueller Hilton (MH) broths, respectively. The bacteria were then pelleted and resuspended in assay buffers to an OD₆₀₀ of 1.0-1.2. Lysin was added to the bacteria with a volume ratio of 1:4 to a final volume of 100 μl. For dose response assays, 10 lysin concentrations of 0.78 to 400 μg/ml were tested. Upon identifying the optimal dose, 25 μg/ml of PlyV12 and 100 μg/ml of LysEF-P10, P10N-V12C, and V12N-P10C were used in the turbidity reduction assays for profiling their optimal pH and salt concentrations. The pH profile experiments were performed after the bacterial cells were resuspended in 20 mM acetate buffer at pH 5 and 6; 20 mM of phosphate buffer at pH 6, 7 and 8; and 20 mM Tris buffer at pH 9 and 10. The activities of the 4 lysins were also characterized under NaCl concentrations ranging from 0 mM to 500 mM. The lytic activity is measured by the reduction in OD₆₀₀ per minute for the first 3 minutes [Yang, H. et al., Sci Rep 7: 1-13 (2017)]. All assays were done with three biological replicates and all buffers were autoclaved before use. The bacterial OD₆₀₀ was measured in a microplate reader (Biotek Synergy 4 Plate Reader) for an hour with occasional shaking at 40-second intervals. Lysin-treated bacteria were serially diluted and plated on BHI and MH agar accordingly for enumeration of colony-forming units (CFU). As a negative control, bacterial strains were treated with equivalent quantity of buffer solutions.

### Flow Cytometry Analysis of Lysin-Treated Bacterial Cells

E. faecium strain C5 was grown to mid-logarithmic phase (OD₆₀₀=0.5) in BHI. The bacteria were then pelleted and resuspended in 10% BHI diluted with 1×PBS, to an OD₆₀₀ of 0.08. Lysins PlyV12 and P10N-V12C were added to the resuspended bacterial cell on a 1:1 ratio such that the starting OD is 0.04 and the final concentrations of lysins are 25 μg/ml (PlyV12) and 100 μg/ml (P10N-V12C), respectively. After 1-hour incubation, 2 μg/ml of Propidium Iodide (PI) was Added and Incubated in the Dark for 15 Minutes at Room Temperature.

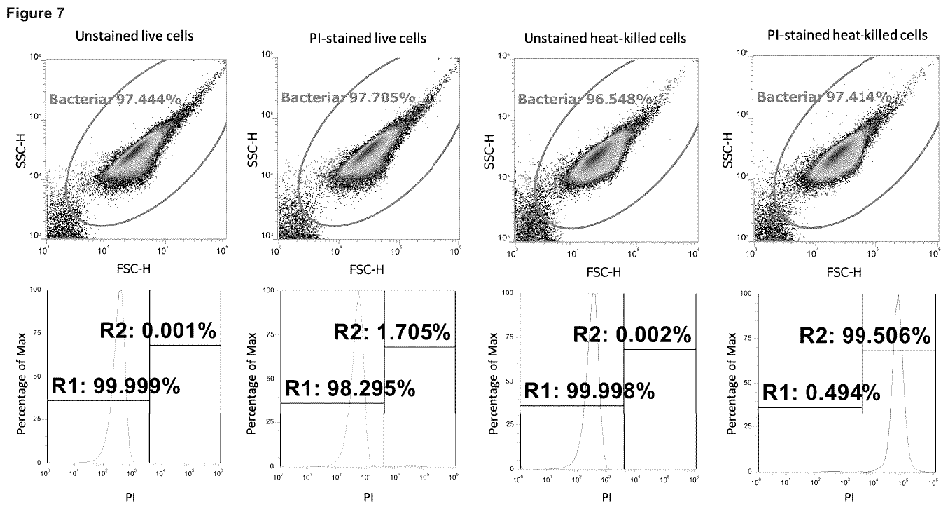

Flow cytometry analysis were performed using Attune NxT 4 lasers and 14 colors system, flat top laser from Thermo Fisher Scientific. Sample delivery utilizing positive-displacement syringe pump for direct volumetric analysis provides concentration measurement without using counting beads. The 96-well U-bottom sample plate was loaded and run with Attune NxT autosampler without prior washing. Total sample volume was 105 μl and acquisition volume was 40 μl. Data acquisition were set at flow rate of 25 μl/min with 2 mixing cycles and 3 rinsing cycles. Total events acquired were 100,000. 25 μl/min speed was the acquisition guideline provided by Thermo Fisher Scientific, with maximum sample concentration of not more than 5.4×10⁷/ml. PI was excited by 561 nm yellow Laser and fluorescent emission were detected at 620/15 band pass emission filter at YL2 PMT detector. The threshold was set at FSC (0.8×1000) and SSC (1.0×1000). Data were collected and analyzed using Attune NxT software. FSC and SSC voltage were set based on unstained healthy bacteria sample. Heat killed bacteria samples were used as a positive control for PI single staining (FIG. 7).

### Bacterial Visualization Using Confocal Microscopy.

Lysin-treated and untreated E. faecium cells were imaged using confocal laser scanning microscopy (CLSM). Propidium Iodine (PI) was added to all sample at a final concentration of 2 μg/mL for 15 minutes in the dark, similar to the flow cytometry experiments. Bacterial cells that are dead or have compromised membrane will uptake PI and appear red under CLSM. Confocal images were acquired on a Zeiss LSM700 CLSM system under bright field light and fluorescence using 40× and 63× oil-immersion objectives. Images were then processed using ZEN software (version 14.0).

### Example 2

### Construct Design and Expression of Recombinant Lysins

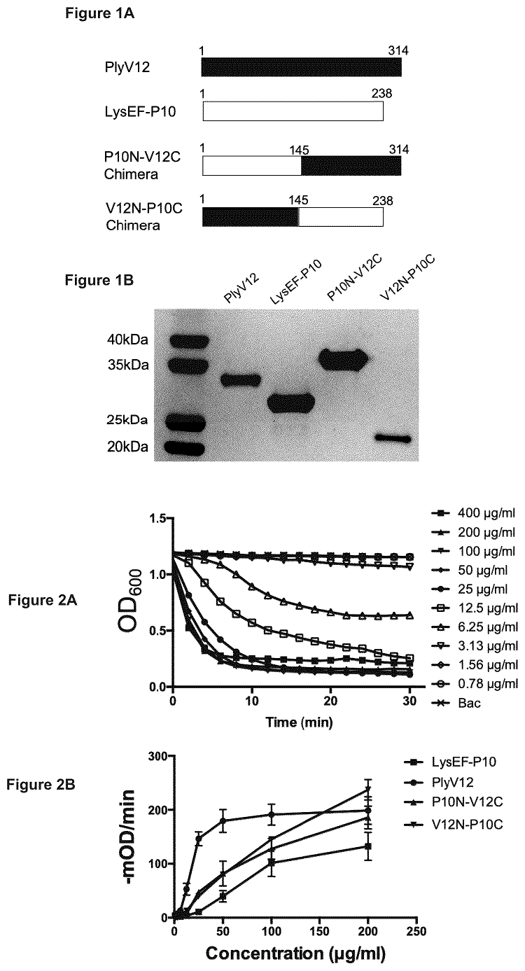

Two native lysins with different lytic spectra were selected as the parent lysins to generate chimeric lysins. LysEF-P10 is a narrow-spectrum lysin that only targets E. faecalis [Cheng, M. et al., Sci Rep 7: 1-15 (2017)], whereas PlyV12 is a broad-spectrum lysin that targets enterococci, staphylococci, and streptococci [Yoong, P. et al., J Bacteriol 186: 4808-4812 (2004)]. Two chimeric lysins, namely P10N-V12C and V12N-P10C, were created by swapping the CBD at the C-terminal domain of the two parent lysins (FIG. 1A). All 4 lysins were successfully expressed and purified (FIG. 1B). The protein bands corresponding to PlyV12 and V12N-P10C are slightly lower than their molecular weights, which is not unusual as such observations have been reported with other lysins [Guo, M. et al., Front Microbiol 8 (2017); Wittmann, J. et al., Microbiology 156: 2366-2373 (2010)]. It is worth noting that the high yield of PlyV12 lysin at 10 mg/L, is contrary to that previously reported [Yoong, P. et al., J Bacteriol 186: 4808-4812 (2004)]. The increased protein yield may be attributed to the recombinant gene with its codon usage optimized for E. coli expression.

### Example 3

### Dose Response and Killing Kinetics of Various Lysins.

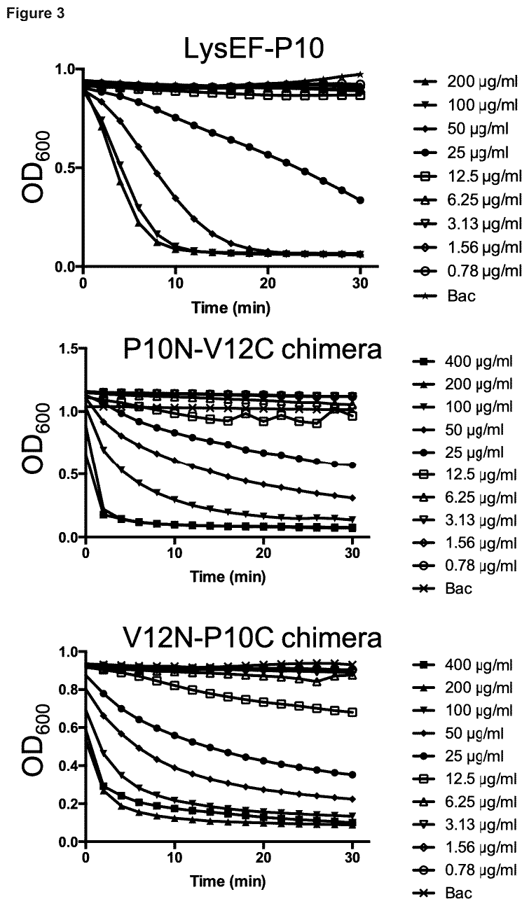

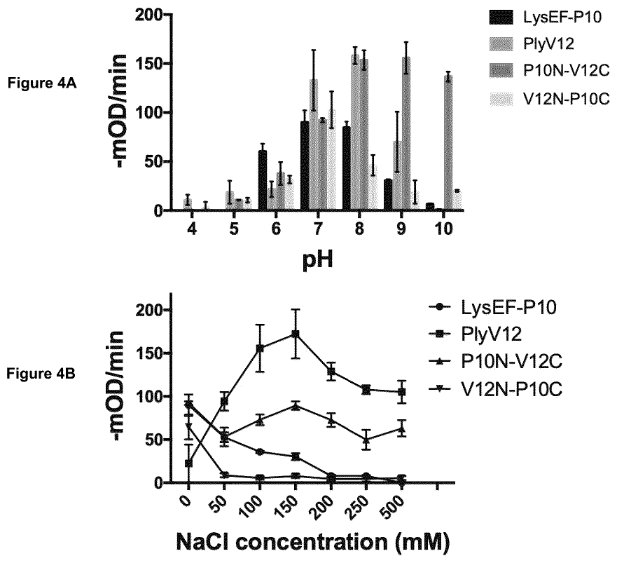

To determine the optimal doses of lysins for subsequent experiments, we measured the lytic activities of all 4 lysins against E. faecalis OG1RF strain by performing turbidity reduction assays. While PlyV12 requires at least 25 μg/ml to achieve >90% reduction in OD₆₀₀ in 30 minutes (FIG. 2A), the other 3 lysins require 100 μg/ml (FIG. 3). At these lysin concentrations, the lytic activities are optimal as indicated by their high lytic rates of over 100-mOD/min (FIG. 2B). Therefore, 25 μg/ml for PlyV12 and 100 μg/ml for LysEF-P10, P10N-V12C and V12N-P10C were selected to further characterize the lysin.

### Example 4

### Lysis Characterization at Various pH and Salt Concentrations

The lytic activities of lysins can vary significantly with pH and salinity [Cheng, M. et al., Sci Rep 7: 1-15 (2017); Yoong, P. et al., J Bacteriol 186: 4808-4812 (2004); Dong, Q. et al., Microb Biotechnol 8: 210-220 (2015); Swift, S. M. et al., FEMS Microbiol Lett 363: 1-8 (2016)]. Therefore, we tested the lysins against E. faecalis OG1RF strain in buffers of pH 4 to pH 10 and a series of salt concentrations ranging from 0 mM to 500 mM NaCl. We observed that P10N-V12C chimeric lysin maintains its lytic activity over a broader pH range, especially at pH 10, than the parent lysins, whereas LysEF-P10 and V12N-P10C share pH 7 as the optimal pH for lytic activity (FIG. 4A). When tested in buffer with increasing salt concentrations, the lytic activities of LysEF-P10 and V12N-P10C reduced significantly with negligible activity in buffers with above 200 mM NaCl (FIG. 4B). However, PlyV12 showed an opposite trend with low activity at 0 mM NaCl and maximum activity at 150 mM NaCl. Although the lytic activity of PlyV12 decreases slightly from 200 mM NaCl onwards, PlyV12 is still active at >100-mOD/min. Interestingly, P10N-V12C chimera exhibit a superior salt tolerance than both of its parent lysins as it retains its lytic activity from 0 mM to 500 mM NaCl.

### Example 5

### Lytic Spectra of Lysins

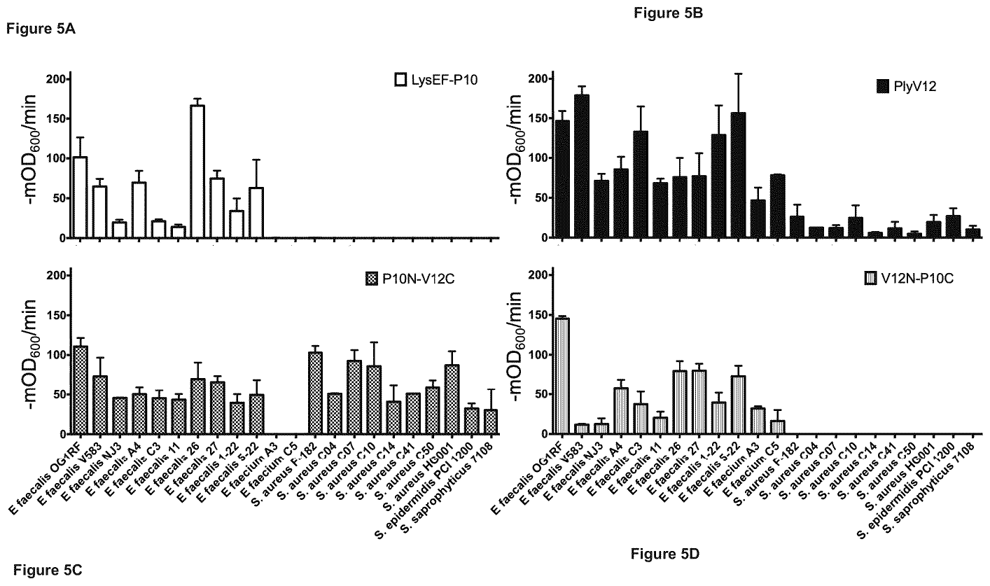

To determine whether the lytic spectrum of a lysin is dictated entirely by its CBD, we swapped the CBD of a species-specific lysin (P10C) with the CBD of a broad-spectrum lysin (V12C), and vice versa. The resulting chimeric lysins, P10N-V12C and V12N-P10C, along with the parent lysins, LysEF-P10 and PlyV12, were tested on 12 enterococcal and 10 staphylococcal strains, including 9 vancomycin-resistant enterococci (VRE) and 7 methicillin-resistance Staphylococcus aureus (MRSA) (Table 2). PlyV12 targets all enterococcal and staphylococcal strains (FIG. 5A). PlyV12 shows a slight preference towards enterococci than staphylococci as illustrated by the higher killing rate on enterococci, which is consistent with Yoong et al [J Bacteriol 186: 4808-4812 (2004)]. LysEF-P10 only targets the E. faecalis strains (FIG. 5B), with a lytic profile that is consistent with the previous study [Cheng, M. et al., Sci Rep 7: 1-15 (2017)].

TABLE 2
Bacterial strains

| | | |
|---|---|---|
| Bacterial strains | Characteristics | Reference or source |

Enterococci

| | | |
|---|---|---|
| E. faecalis OG1RF | Rifampicin resistant | ATCC 47077, * |
| E. faecalis V583 | Vancomycin resistant | ATCC 700802, \hat{ } |
| E. faecalis NJ3 | Vancomycin resistant | ATCC 51299 |
| E. faecalis A4 | Isolate from healthy child gut | WUSTL, # |
| E. faecalis C3 | Isolate from healthy child gut | WUSTL, # |
| E. faecalis 11 | Non-VRE clinical wound isolate | TTSH |
| E. faecalis 26 | Non-VRE clinical wound isolate | TTSH |
| E. faecalis 27 | Non-VRE clinical wound isolate | TTSH |
| E. faecalis 1-22 | VRE clinical blood isolate | WUSTL, # |
| E. faecalis 5-22 | VRE clinical blood isolate | WUSTL, # |
| E. faecium A3 | Isolate from healthy child gut | WUSTL, # |
| E. faecium C5 | Isolate from healthy child gut | WUSTL, # |

Staphylococci

| | | |
|---|---|---|
| S. aureus F-182 | Methicillin resistant | ATCC 43300 |
| S. aureus C04 | MRSA clinical wound isolate | TTSH |
| S. aureus C07 | MRSA clinical wound isolate | TTSH |
| S. aureus C10 | MRSA clinical wound isolate | TTSH |
| S. aureus C14 | MRSA clinical wound isolate | TTSH |
| S. aureus C41 | MRSA clinical wound isolate | TTSH |
| S. aureus C50 | MRSA clinical wound isolate | TTSH |
| S. aureus HG001 | Methicillin susceptible | ! |
| S. epidermidis | Non-pathogenic commensal | ATCC 12228 |
| PCI 1200 | | |
| S. saprophyticus 7108 | Wild-type strain | @ |
| S. hominis SK119 | Isolate from healthy skin | BEI resources |
| S. hominis C80 | Clinical isolate from sputum | BEI resources |

WUSTL: Washington University in St. Louis, USA.
TTSH: Tan Tock Seng Hospital, Singapore.
* Dunny, G. M. et al., Proc Natl Acad Sci 75:3479-3483 (1978)
\hat{ } Paulsen, I. T. et al., Science 299:2071-4 (2003)
# Denno, D. M. et al., Clin Infect Dis 55:897-904 (2012)
! Caldelari, I. et al., Genome Announc 5 (2017)
@ Gatemann, S. et al., FEMS Microbiol Lett 47:179-185 (1988)

Swapping the broad-spectrum CEO from PlyV12 with a CEO from the narrow-spectrum LysEF-P10 lysin, the V12N-P10C chimera kills both E. faecalis and E. faecium, but it does not target staphylococci (FIG. 50), suggesting the CEO is crucial in distinguishing bacteria of different genera. For the chimeric lysin with the CEO of PlyV12, P10N-V12C can target all staphylococcal strains and all E. faecalis strains (FIG. 50). However, it does not kill E. faecium. This was surprising because a previously-reported chimeric lysin P187N-V12C, which also carries the CEO of PlyV12, was shown to lyse all of the tested streptococcal, staphylococcal, and enterococcal strains, including E. faecium [Dong, Q. et al., Microb Biotechnol 8: 210-220 (2015)]. Considering that the only difference between P187N-V12C and our P10N-V12C chimeric lysin is the catalytic domain, we deduced that the catalytic domain in P10N-V12C is responsible for distinguishing E. faecium from E. faecalis.

### Example 6

The Effect of P10N-V12C Lysin on E. faecium

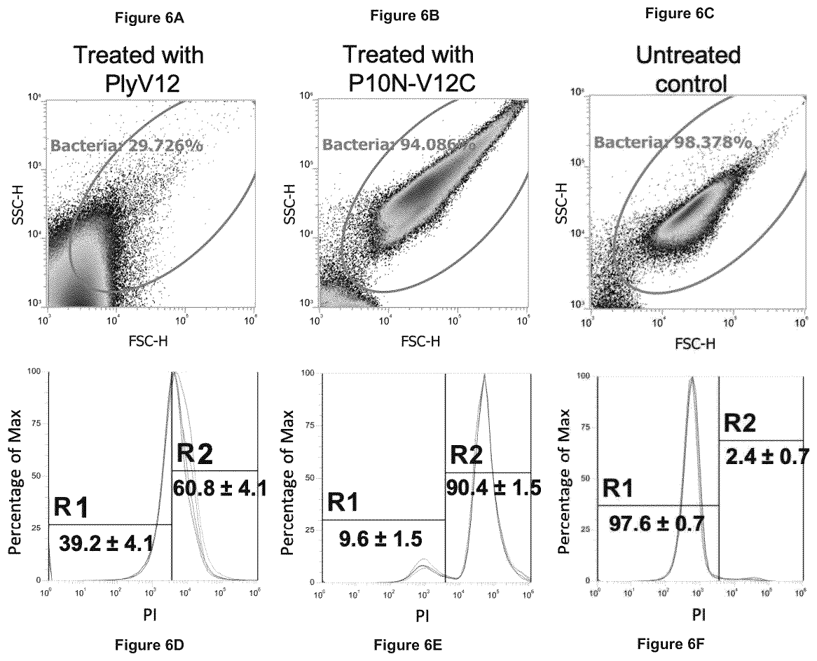

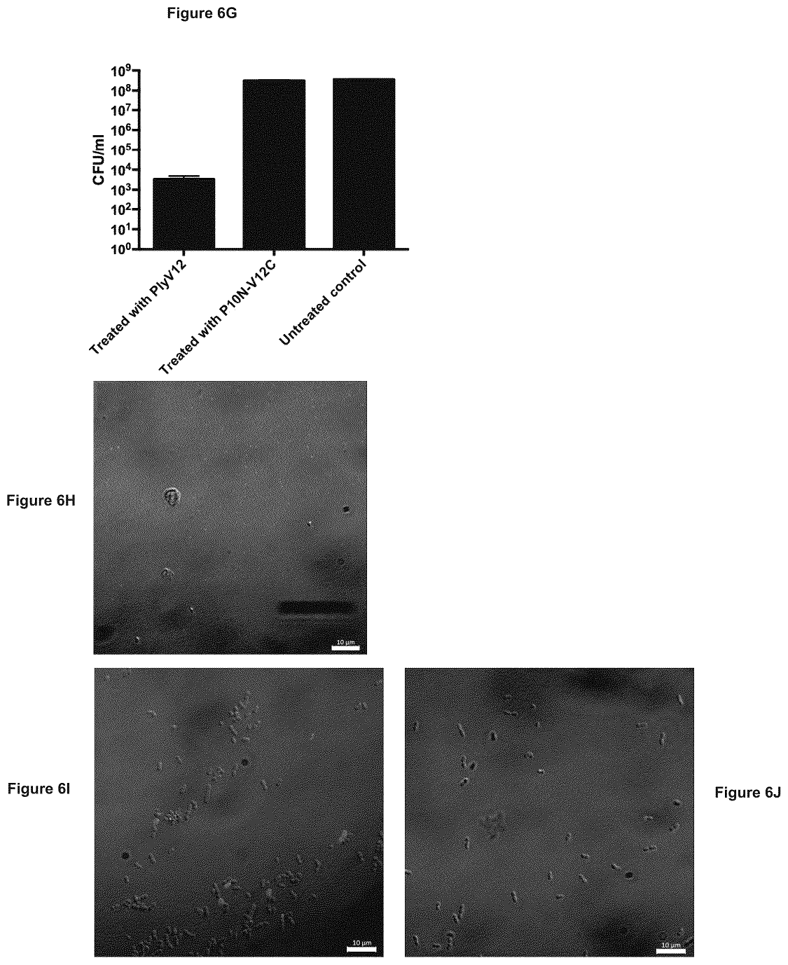

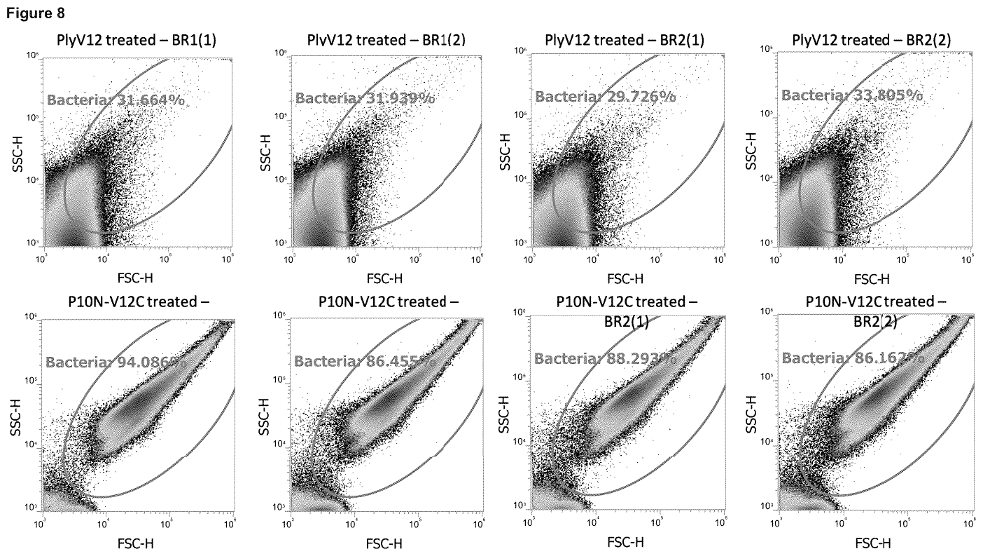

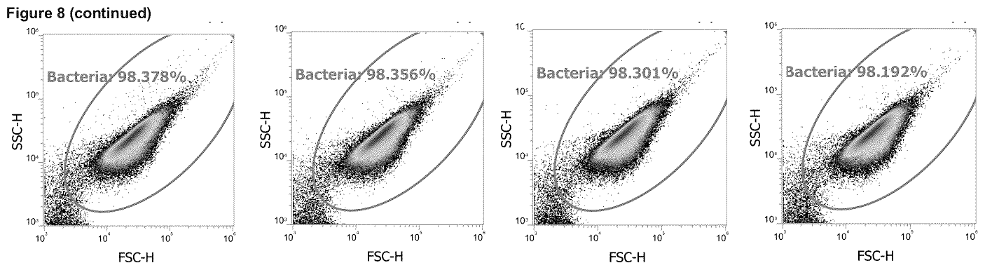

To investigate why P10N-V12C does not kill E. faecium, despite carrying a broad-spectrum CBD, lysin-treated E. faecium cells were further analyzed using flow cytometry and confocal microscopy. We first observed that PlyV12 effectively lysed most E. faecium cells as reflected by the low number of intact cells (FIG. 6A) and the 5-log CFU/ml reduction (FIG. 6G). The forward-scattered (FSC) and side-scattered (SSC) values of the PlyV12-treated cells (FIG. 6A) were drastically reduced compared to the untreated control (FIG. 6C). This reduction can be attributed to the cell debris and aggregates formed upon cell lysis. Additionally, significantly fewer bacterial cells were visible by confocal microscopy. Instead of individual intact cells, aggregates of presumably dead cells were observed upon PlyV12 treatment (FIG. 6H). By contrast, the P10N-V12C-treated E. faecium cells (FIGS. 6B and 8) were more elongated and slightly larger in overall size based on their FSC/SSC profiles compared to the untreated controls (FIG. 6C). These cells are still viable as the CFU count is similar to the control (FIG. 6G). However, most bacterial cells treated with P10N-V12C show significant uptake of PI dye as measured by flow cytometry (FIG. 6E) and observed in the confocal images (FIG. 6I), signifying the cell membrane has been compromised. The membrane is likely compromised by the lysins bound to the cell wall as the CBD of PlyV12 is known to bind to E. faecium [Dong, Q. et al., Microb Biotechnol 8: 210-220 (2015)]. Despite binding to the cell wall and compromising the membrane with its CBD, the chimeric lysin P10N-V12C does not lyse and kill E. faecium cells because its species-specific CD does not hydrolyze the peptidoglycan of E. faecium.

### Example 7

Screening and Isolation of Chimeric Lysins from a Library

Further chimeric lysins were obtained by creating a library using Golden Gate assembly. Chimeric lysins generated with this method have a short linker peptide sequence between the lysin domains.

### Construction of Lysin Library

A library of 100 chimeric lysins was generated by shuffling the catalytic and cell-wall binding domains of 10 naturally occurring lysins using a cloning technique called Golden Gate assembly. The vector used is pNIC28-Bsa4 with Bsal restriction site that enables seamless cloning. The respective domains were identified by integrating the information from a list of sequence and structure prediction software, namely BLAST, JPRED, I-TASSER, and RaptorX. The nucleotide sequences of each domain were codon-optimized to improve the efficiency of soluble expression in E. coli. The amino acid and nucleotide sequences of the domains are represented by LKCHAPV12 (SEQ ID NOs: 9 and 10), V12NM3 (SEQ ID Nos: 13 and 14), LKNM3 (SEQ ID Nos: 15 and 16). The domains of each chimera were fused with a GSSG peptide linker. This GSSG linker was added between the two domains because it is a partial byproduct of Golden Gate assembly. Furthermore, the addition of a short linker has been previously shown to improve soluble expression as well as antibacterial activity [Yang, H. et al. Antimicrob. Agents Chemother. (2020) 64:e01610-19. doi: 10.1128/AAC.01610-19].

### Medium-Throughput Expression, Screening and Isolation

To screen the library for lysins with high activity against BO-causing bacteria, a medium-throughput protein expression method was developed with reference to Duyvejonck et al. (Duyvejonck L, et al., Antibiotics (Basel). (2021) 10(3):293. doi: 10.3390/antibiotics10030293).

This method enables 200-400 variants to be tested at one time.

The protocol of the medium-throughput assay is as follows: First, the gene library of 100 chimeric lysins was transformed in E. coli BL21 (DE3) T1R strain and spread on an LB agar plate with the selection antibiotics, namely kanamycin and chloramphenicol. Upon overnight incubation, the ˜200 colonies were individually picked and inoculated into deep-well 96-well plates containing LB and the selection antibiotics for an overnight incubation with constant shaking at 700 rpm. 10 μl of the overnight culture was used to inoculate another set of deep-well 96-well plates containing autoinduction media and the selection antibiotics, namely kanamycin and chloramphenicol. The 96-well plates were incubated at room temperature for 3 days to enable protein overexpression. The original 96-well plate with the uninduced overnight culture was made into glycerol stock and kept in −80° C. for downstream isolation purpose. Upon the 3-day incubation, the plates were spun down to remove media and resuspended with 1×PBS. The expressed protein was obtained by lysing the cells using a freeze-thaw cycle method, namely putting the plate in liquid nitrogen and heated water bath for 7 times. The plates were spun down to remove the cell debris and the supernatant was transferred to another 96-well plate where it is ready to be tested for its activity.

The main difference in methodology is that we replaced the cell lysis method with a safer alternative. Duyvejonck et al. uses chloroform gas, a toxic and corrosive chemical, to lyse the cells whereas we use freeze-thaw method which uses liquid nitrogen and warm water to rapidly freeze and thaw the cells for 5-7 cycles, ultimately lysing the cells and releasing the intracellularly expressed protein to the supernatant.

Furthermore, we used different screening assays compared to Duyvejonck et al. We performed turbidity reduction assay and lysate clearance assay. The turbidity reduction assay is a routine assay to evaluate the activity of lysins targeting Gram-positive bacteria [Yoong, P. et al., J Bacteriol 186:4808-4812 (2004); Binte Muhammad Jai, H. S., et al., Front. Microbiol. 11: 2868 (2020)]. The lysate clearance assay was developed with reference to Raz et al [Raz A, et al., Antimicrob Agents Chemother. 2019 63(7):e00024-19. doi: 10.1128/AAC.00024-19], which was originally intended to test lysin's activity against Gram-negative bacteria. 3 bacterial species were tested on the turbidity reduction assay, namely Staphylococcus hominis, Staphylococcus epidermidis, and Corynebacterium striatum. For lysate clearance assay, only S. epidermidis was tested as it is an assay to test for catalytic activity as a proxy to gauge the expressed protein is functional.

For lysate clearance assay, 2.5 μl of lysate or purified proteins were spotted on the autoclaved bacterial agar of S. epidermidis. To prepare this bacterial agar, S. epidermidis was grown overnight in 1 L of LB, harvested and resuspended with 250 ml of 1×PBS. 1.75 g of agarose (0.7% agarose) was added to the resuspended solution before autoclaving and kept at 4° C. until use. The protocol of turbidity reduction assay is described in the next sections.

### Example 8

Engineered Chimeric Lysins that can Target Body Odor-Causing Bacteria

Table 3 shows a summary of the killing activities of 4 engineered lysins, LKCHAPV12, P10N-V12C, V12NM3 and LKNM3, when compared to PlyV12, the parent lysin that served as positive control. Lysin LKCHAPV12 exhibit good killing activity on all staphylococcal species tested and more importantly, LKCHAPV12 remains active in human sweat, whereas PlyV12 lost its activity entirely. Another 16 engineered lysins were tested but showed little activity in this assay (data not shown).

TABLE 3
result table for engineered lysins #1-4 against positive control PlyV12

| | Killing activity at 100 μg/ml | | | | | | | | | |
|---|---|---|---|---|---|---|---|---|---|---|
| | in PBS | | | | | | | | | |
| | S. hominis | | | | | | In sweat | | | |
| Lysins | SK119 | | S. epidermidis | | S. aureus | | S. hominis | | S. epidermidis | |
| PlyV12 | 25 | ++ | 90 | +++ | 47 | ++ | 0 | − | 9 | + |
| LKCHAPV12 | 39 | ++ | 24.5 | ++ | 49.3 | ++ | 7.3 | + | 25.8 | ++ |
| P10N-V12C | 13.7 | ++ | 40 | ++ | 53.8 | +++ | 1.3 | + | 13.8 | ++ |
| V12NM3 | 5.8 | + | 27.3 | ++ | 25 | ++ | 0 | − | 0 | − |
| LKNM3 | 7.3 | + | 15.5 | ++ | 22.5 | ++ | 0 | − | 2.3 | + |

The activity levels of the lysins against various bacteria at 100 ug/ml are also shown as −, +, ++, +++
− refers to no killing
+ refers to -mOD/min below 10
++ refers to -mOD/min between 10 and 50
+++ refers to -mOD/min above 50

Additionally, P10N-V12C also remains active against S. hominis in human sweat. 4 out of the 5 lysins tested are active against S. epidermidis in human sweat while only LKCHAPV12 and P10N-V12C are active against S. hominis in human sweat, suggesting S. hominis is harder to kill in human sweat. Another 2 engineered lysins, V12NM3 and LKNM3, are active against S. hominis, S. epidermidis, and S. aureus in PBS but mostly lost their activity when they are in the sweat environment.

### Example 9

### Combination of Lysins can Achieve Enhanced Bacterial Killing

Enhanced killing was achieved when combining an engineered lysin (LKCHAPV12) and a native lysin (PlyV12) (Table 4).

TABLE 4
result table illustrating synergistic activity between
PlyV12 and LKCHAPV12 on Staphylococcus hominis.

| % of OD reduction after 1 hour treatment in turbidity reduction assay | | | | | | | | | | |
|---|---|---|---|---|---|---|---|---|---|---|
| PlyV12 + | PlyV12 | | | | | | | | | |
| LKCHAPV12 | (μg/ml) | 1 | 2 | 3 | 4 | 5 | 6 | 7 | 8 | |
| A | 100 | 21 | 68 | 86 | 93 | 91 | 82 | 77 | 64 | |
| B | 50 | 0 | 73 | 86 | 92 | 90 | 86 | 77 | 72 | |
| C | 25 | 0 | 79 | 87 | 86 | 91 | 88 | 76 | 71 | |
| D | 12.5 | 0 | 54 | 34 | 88 | 92 | 90 | 80 | 70 | |
| E | 6.25 | 0 | 24 | 75 | 81 | 95 | 90 | 83 | 77 | |
| F | 3.125 | 0 | 13 | 51 | 73 | 88 | 85 | 79 | 78 | |
| G | 1.5625 | 0 | 8 | 28 | 63 | 86 | 82 | 80 | 70 | |
| H | 0 | 0 | 0 | 28 | 53 | 69 | 73 | 57 | 69 | |
| | | 0 | 1.6 | 3.125 | 6.25 | 12.5 | 25 | 50 | 100 | LKCHAPV12 |
| | | | | | | | | | | (μg/ml) |

Notably, >80% killing can be achieved by combining 6.25 μg/ml of PlyV12 and LKCHAPV12, respectively, whereas it would require >100 μg/ml of each lysin to achieve individually. Such synergistic effect will lower the effective materials needed, thus translating to cost reduction. The apparent lower percentage of OD reduction at higher concentrations (50-100 μg/ml) is a result of formation of cell clumps. The cultures in those wells were clear, indicating >85% cell lysis and death.

### SUMMARY

With the emergence of multidrug-resistant bacteria, including VRE and MRSA, there is an urgent need to develop new antibacterial therapeutics.

In this study, we constructed chimeric lysins by swapping the CBD of two parent lysins with different properties. We found that domain-swapping affects the functional pH and salinity tolerance of the two chimeric lysins. P10N-V12C outperformed its parent lysins by maintaining high lytic activities across a broader range of pH and salt concentrations. However, the other chimeric lysin, V12N-P10C, has limited pH range with diminished activity in the presence of salt. These observations show the importance of CBD in determining the optimal pH and salt concentration where a lysin is most active at. As observed in lysin LKCHAPV12, the chimeric lysin showed superior activity in human sweat compared to its parent lysin PlyV12, thereby illustrating the advantages of chimeric lysins.

The two parent lysins selected in this study have different lytic spectra: LysEF-P10 only targeting E. faecalis and PlyV12 targeting multiple genera. Upon swapping the CBD of LysEF-P10, the lytic spectrum of V12N-P10C was narrowed as it only targets enterococci. Conversely, P10N-V12C exhibits a broadened lytic spectrum with the CBD of PlyV12. Interestingly, although P10N-V12C now targets staphylococcal strains, it retains the species-specificity feature of LysEF-P10 on E. faecalis and does not target E. faecium. These data suggest that the CD also contributes, albeit to a lesser extent compared to the CBD, to the final lytic spectrum of a chimeric lysin.

Flow cytometry and confocal microscopy revealed that the E. faecium cells that have been treated with P10N-V12C have a compromised membrane, but the cells remain intact and viable. It was previously shown that GFP-fused CBD of PlyV12 binds to staphylococcal and enterococcal cells, including E. faecium [Dong, Q. et al., Microb Biotechnol 8:210-220 (2015)], therefore the CBD of P10N-V12C is expected to bind to the membrane of E. faecium. This could explain the slightly larger and elongated E. faecium cells upon exposure of P10N-V12C. Such binding event would disrupt the bacterial membrane to an extent that enables the PI uptake of the cells. However, the treated cells were not lysed and remained viable as the CD of P10N-V12C does not hydrolyze the peptidoglycan of E. faecium.

The present invention provides species specificity to an otherwise, broad-spectrum lysin by swapping its catalytic domain with that of a species-specific lysin. The resulting chimeric lysin P10N-V12C selectively targets E. faecalis over E. faecium while it remains active against all staphylococcal strains tested. It possesses a high lytic activity across a broader range of pH and salt concentrations than any of the parent lysins. While CBD swapping helps to extend the lytic spectrum such that multiple bacterial genera can be targeted, choosing the correct CD to fuse with the CBD is important to achieve species specificity in a particular genus. With more understanding of the roles of both CD and CBD, novel lysins can be tailor-made to develop a highly-targeted antimicrobial therapy.

Not every chimeric lysin is suitable to be used for commercial applications. Out of the 100 possible chimeric lysins we screened, some lost their activity in moderate-to-high salinity environment, and some did not have soluble expression. Here we have identified 4 engineered lysins with the potential to be used in treating body odor, atopic dermatitis (eczema), bacteremia, as well as healing wound with bacterial infections. These lysins can be used alone, or in combination to achieve additive or synergistic effect. For example, PlyV12 and LKCHAPV12 exhibit synergy in killing Staphylococcal when both are used in combination.

### REFERENCES

1. Binte Muhammad Jai, H. S., Dam, L. C., Tay, L. S., Koh, J. J. W., Loo, H. L., Kline, K. A. and Goh, B. C. (2020) Engineered Lysins With Customized Lytic Activities Against Enterococci and Staphylococci. Front. Microbiol., 11: 2868.
2. Broendum, S. S., Buckle, A. M. & McGowan, S. Catalytic diversity and cell wall binding repeats in the phage-encoded endolysins. Mol. Microbiol. 110, 879-896 (2018).
3. Caldelari, I. et al. Complete Genome Sequence and Annotation of the Staphylococcus aureus Strain HG001. Genome Announc. 5, (2017).
4. Cheng, M. et al. Endolysin LysEF-P10 shows potential as an alternative treatment strategy for multidrug-resistant Enterococcus faecalis infections. Sci. Rep. 7, 1-15 (2017).
5. Denno, D. M. et al. Diarrhea Etiology in a Pediatric Emergency Department: A Case Control Study. Clin. Infect. Dis. 55, 897-904 (2012).
6. Deutsch, S.-M., Guezenec, S., Piot, M., Foster, S. & Lortal, S. Mur-LH, the Broad-Spectrum Endolysin of Lactobacillus helveticus Temperate Bacteriophage φ-0303. Appl. Environ. Microbiol. 70, 96-103 (2004).
7. Diez-Martinez, R. et al. Improving the Lethal Effect of Cpl-7, a Pneumococcal Phage Lysozyme with Broad Bactericidal Activity, by Inverting the Net Charge of Its Cell Wall-Binding Module. Antimicrob. Agents Chemother. 57, 5355-5365 (2013).
8. Dong, Q. et al. Construction of a chimeric lysin Ply187N-V12C with extended lytic activity against staphylococci and streptococci. Microb. Biotechnol. 8, 210-220 (2015).
9. Dunny, G. M., Brown, B. L. & Clewell, D. B. Induced cell aggregation and mating in Streptococcus faecalis: evidence for a bacterial sex pheromone. Proc. Natl. Acad. Sci. 75, 3479-3483 (1978).
10. Duyvejonck L, Gerstmans H, Stock M, Grimon D, Lavigne R, Briers Y. Rapid and High-Throughput Evaluation of Diverse Configurations of Engineered Lysins Using the VersaTile Technique. Antibiotics (Basel). 2021 Mar. 11; 10(3):293. doi: 10.3390/antibiotics10030293.
11. Fischetti, V. A. Bacteriophage lysins as effective antibacterials. Curr. Opin. Microbiol. 11, 393-400 (2008).
12. Fischetti, V. A. Bacteriophage endolysins: A novel anti-infective to control Gram-positive pathogens. Int. J. Med. Microbiol. 300, 357-362 (2010).
13. Fischetti, V. A. Development of phage lysins as novel therapeutics: A historical perspective. Viruses 10, (2018).
14. Gatermann, S., Marre, R., Heesemannn, J. & Henkel, W. Hemagglutinating and adherence properties of Staphylococcus saprophyticus: epidemiology and virulence in experimental urinary tract infection of rats. FEMS Microbiol. Lett. 47, 179-185 (1988).
15. Gilmer, D. B., Schmitz, J. E., Euler, C. W. & Fischetti, V. A. Novel Bacteriophage Lysin with Broad Lytic Activity Protects against Mixed Infection by Streptococcus pyogenes and Methicillin-Resistant Staphylococcus aureus. Antimicrob. Agents Chemother. 57, 2743-2750 (2013).
16. Guo, M. et al. A Novel Antimicrobial Endolysin, LysPA26, against Pseudomonas aeruginosa. Front. Microbiol. 8, (2017).
17. Hermoso, J. A., Garcia, J. L. & Garcia, P. Taking aim on bacterial pathogens: from phage therapy to enzybiotics. Curr. Opin. Microbiol. 10, 461-472 (2007).
18. Loessner, M. J., Kramer, K., Ebel, F. & Scherer, S. C-terminal domains of Listeria monocytogenes bacteriophage murein hydrolases determine specific recognition and high-affinity binding to bacterial cell wall carbohydrates. Mol. Microbiol. 44, 335-349 (2002).
19. Lóez, R., Garcia, E., Garcia, P. & Garcia, J. L. The Pneumococcal Cell Wall Degrading Enzymes: A Modular Design to Create New Lysins? Microb. Drug Resist. 3, 199-211 (1997).
20. Love, M. J., Abeysekera, G. S., Muscroft-Taylor, A. C., Billington, C. & Dobson, R. C. J. On the catalytic mechanism of bacteriophage endolysins: Opportunities for engineering. Biochim. Biophys. Acta-Proteins Proteomics 1868, 140302 (2020).
21. Low, L. Y., Yang, C., Perego, M., Osterman, A. & Liddington, R. Role of net charge on catalytic domain and influence of cell wall binding domain on bactericidal activity, specificity, and host range of phage lysins. J. Biol. Chem. 286, 34391-34403 (2011).
22. Maciejewska, B., Olszak, T. & Drulis-Kawa, Z. Applications of bacteriophages versus phage enzymes to combat and cure bacterial infections: an ambitious and also a realistic application? Appl. Microbiol. Biotechnol. 102, 2563-2581 (2018).
23. Nelson, D., Loomis, L. & Fischetti, V. A. Prevention and elimination of upper respiratory colonization of mice by group A streptococci by using a bacteriophage lytic enzyme. Proc. Natl. Acad. Sci. 98, 4107-4112 (2001).
24. Nelson, D. C. et al. Endolysins as Antimicrobials. in Advances in Virus Research (2012). doi:10.1016/B978-0-12-394438-2.00007-4
25. O'Flaherty, S., Coffey, A., Meaney, W., Fitzgerald, G. F. & Ross, R. P. The Recombinant Phage Lysin LysK Has a Broad Spectrum of Lytic Activity against Clinically Relevant Staphylococci, Including Methicillin-Resistant Staphylococcus aureus. J. Bacteriol. 187, 7161-7164 (2005).
26. Oliveira, H. et al. Molecular Aspects and Comparative Genomics of Bacteriophage Endolysins. J. Virol. 87, 4558-4570 (2013).
27. Oliveira, H., São-José, C. & Azeredo, J. Phage-derived peptidoglycan degrading enzymes: Challenges and future prospects for in vivo therapy. Viruses 10, (2018).
28. Paulsen, I. T. et al. Role of mobile DNA in the evolution of vancomycin-resistant Enterococcus faecalis. Science 299, 2071-4 (2003).
29. Raz A, Serrano A, Hernandez A, Euler C W, Fischetti V A. Isolation of Phage Lysins That Effectively Kill Pseudomonas aeruginosa in Mouse Models of Lung and Skin Infection. Antimicrob Agents Chemother. 2019 Jun. 24; 63(7):e00024-19. doi: 10.1128/AAC.00024-19.
30. Schleifer, K. H. & Kandler, O. Peptidoglycan types of bacterial cell walls and their taxonomic implications. Bacteriol. Rev. 36, 407-77 (1972).
31. Schmelcher, M., Donovan, D. M. & Loessner, M. J. Bacteriophage endolysins as novel antimicrobials. Future Microbiol. 7, 1147-1171 (2012).
32. Schmelcher, M., Tchang, V. S. & Loessner, M. J. Domain shuffling and module engineering of Listeria phage endolysins for enhanced lytic activity and binding affinity. Microb. Biotechnol. 4, 651-662 (2011).
33. Swift, S. M. et al. The endolysin from the Enterococcus faecalis bacteriophage VD13 and conditions stimulating its lytic activity. FEMS Microbiol. Lett. 363, 1-8 (2016).
34. Velez, R. & Sloand, E. Combating antibiotic resistance, mitigating future threats and ongoing initiatives. J. Clin. Nurs. 25, 1886-1889 (2016).
35. Ventola, C. L. The antibiotic resistance crisis: part 1: causes and threats. P T 40, 277-83 (2015).
36. Vermassen, A. et al. Cell wall hydrolases in bacteria: Insight on the diversity of cell wall amidases, glycosidases and peptidases toward peptidoglycan. Front. Microbiol. 10, (2019).
37. Wittmann, J., Eichenlaub, R. & Dreiseikelmann, B. The endolysins of bacteriophages CMP1 and CN77 are specific for the lysis of Clavibacter michiganensis strains. Microbiology 156, 2366-2373 (2010).
38. Yang, H., Yu, J. & Wei, H. Engineered bacteriophage lysins as novel anti-infectives. Front. Microbiol. 5, 1-6 (2014).
39. Yang, H. et al. Novel Chimeric Lysin with High-Level Antimicrobial Activity against Methicillin-Resistant Staphylococcus aureus In Vitro and In Vivo. Antimicrob. Agents Chemother. 58, 536-542 (2014).
40. Yang, H., Zhang, H., Wang, J., Yu, J. & Wei, H. A novel chimeric lysin with robust antibacterial activity against planktonic and biofilm methicillin-resistant Staphylococcus aureus. Sci. Rep. 7, 1-13 (2017).
41. Yang, H. et al. ClyJ Is a Novel Pneumococcal Chimeric Lysin with a Cysteine- and Histidine-Dependent Amidohydrolase/Peptidase Catalytic Domain. Antimicrob. Agents Chemother. (2019) 63:e02043-18. doi: 10.1128/AAC.02043-18.
42. Yang, H. et al. Linker Editing of Pneumococcal Lysin ClyJ Conveys Improved Bactericidal Activity. Antimicrob. Agents Chemother. (2020) 64, :e01610-19. doi: 10.1128/AAC.01610-19.
43. Yoong, P., Schuch, R., Nelson, D. & Fischetti, V. A. Identification of a Broadly Active Phage Lytic Enzyme with Lethal Activity against Antibiotic-Resistant Enterococcus faecalis and Enterococcus faecium. J. Bacteriol. 186, 4808-4812 (2004).
44. Zimmer, M., Sattelberger, E., Inman, R. B., Calendar, R. & Loessner, M. J. Genome and proteome of Listeria monocytogenes phage PSA: an unusual case for programmed+1 translational frameshifting in structural protein synthesis. Mol. Microbiol. 50, 303-317 (2003).
45. Zou, G. et al. Functional Analysis of Two Cavities in Flavivirus NS5 Polymerase. J. Biol. Chem. 286, 14362-14372 (2011).

1. A chimeric bacteriophage lysin comprising a catalytic domain of a first phage lysin and a binding domain of a second phage lysin, wherein the chimeric lysin has killing activity against a plurality of Staphylococcus species, wherein the lysin comprises an amino acid sequence set forth in:
a) SEQ ID NO: 11, or a variant thereof wherein said variant is capable of killing staphylococci;
b) amino acids 1-165 and 170-338 set forth in SEQ ID NO: 9, or a variant thereof wherein said variant is capable of killing staphylococci;
c) amino acids 1-145 and 150-242 set forth in SEQ ID NO: 13, or a variant thereof wherein said variant is capable of killing staphylococci;
d) amino acids 1-165 and 170-262 set forth in SEQ ID NO: 15, or a variant thereof wherein said variant is capable of killing staphylococci.

2. The chimeric lysin of claim 1, wherein the variant comprises a sequence having at least 85%, at least 90% or at least 95% sequence identity to the amino acid sequence set forth in a) SEQ ID NO: 11; b) SEQ ID NO: 9; c) SEQ ID NO: 13; or d) SEQ ID NO: 15.

3. The chimeric lysin of claim 1, wherein the said first and second binding domains are fused by a linker peptide.

4. The chimeric lysin of claim 3, wherein the linker peptide has the amino acid sequence set forth in amino acids 166-169 of SEQ ID NO: 9.

5. The chimeric lysin of claim 1, wherein:
a) the chimeric lysin has the amino acid sequence set forth in SEQ ID NO: 11;
b) the chimeric lysin has the amino acid sequence set forth in SEQ ID NO: 9;
c) the chimeric lysin has the amino acid sequence set forth in SEQ ID NO: 13;
d) the chimeric lysin has the amino acid sequence set forth in SEQ ID NO: 15.

6. The chimeric lysin of claim 1, wherein:
i) the chimeric lysin set forth in a) also has killing activity against E. faecalis and/or S. hominis and S. epidermidis in human sweat;
ii) the chimeric lysin set forth in b) has killing activity against hominis and S. epidermidis in human sweat;
iii) the chimeric lysin set forth in d) has killing activity against S. epidermidis in human sweat.

7. The chimeric lysin of claim 1, wherein the chimeric lysin in a) has killing activity against methicillin-resistant and methicillin-sensitive strains of S. aureus.

8. The chimeric lysin of claim 1, wherein the chimeric lysin in a) has killing activity against Rifampicin resistant strains of E. faecalis and Vancomycin resistant strains of E. faecalis.

9. The chimeric lysin of claim 1, wherein the chimeric lysin in a) has killing activity over a pH range of about 4-10 and/or over a salt concentration of about 0-500 mM NaCl.

10. An isolated polynucleotide molecule encoding a chimeric lysin of claim 1.

11. The isolated polynucleotide molecule of claim 10, wherein:
i) the nucleic acid sequence in a) has at least 85% sequence identity to SEQ ID NO: 12;
ii) the nucleic acid sequence in b) has at least 85% sequence identity to SEQ ID NO: 10;
iii) the nucleic acid sequence in c) has at least 85% sequence identity to SEQ ID NO: 14;
iv) the nucleic acid sequence in d) has at least 85% sequence identity to SEQ ID NO: 16;
due to the degeneracy of the genetic code.

12. A composition comprising one or more chimeric lysins of claim 1.

13. The composition of claim 12, further comprising an antibiotic and/or a native lysin such as PlyV12 (SEQ ID NO: 17) or LysEF-P10 (SEQ ID NO: 19).

14. The composition of claim 13, wherein the chimeric lysin is LKCHAPV12 (SEQ ID NO: 9) and the native lysin is PlyV12 (SEQ ID NO: 17).

15. The composition of claim 12, for sanitizing or decontaminating porous or non-porous surfaces.

16.-18. (canceled)

19. A method of prophylaxis or treatment of a staphylococcal infection in a mammal, comprising administering to said mammal one or more chimeric lysins of claim 1.

20. The method of claim 19, wherein the staphylococcal infection causes body odor in a human and the one or more chimeric lysins are selected from claim 1.

21. A method for decontaminating inanimate surfaces suspected of containing infectious bacteria, comprising treatment of said surfaces with a bactericidal or bacteriostatically effective amount of at least one chimeric bacteriophage lysin of claim 1.
